(19) United States
(12) Patent Application Publication
(10) Pub. No.: US 2025/0085248 A1
(43) Pub. Date: Mar. 13, 2025

(54) RESISTIVE HYDROGEN SENSOR COMPRISING SENSING LAYER HAVING SEMICONDUCTING SINGLE-WALLED CARBON NANOTUBES, AND MANUFACTURING METHOD THEREFOR

(71) Applicant: POSTECH RESEARCH AND BUSINESS DEVELOPMENT FOUNDATION, Pohang-si (KR)

(72) Inventors: Yong-Young NOH, Pohang-si (KR); Gi-Seong RYU, Pohang-si (KR)

(21) Appl. No.: 18/291,633

(22) PCT Filed: Jan. 20, 2023
(86) PCT No.: PCT/KR2023/001021

§ 371 Date: Jan. 24, 2024

(30) Foreign Application Priority Data
Jan. 25, 2022 (KR) 10-2022-0010508
Jan. 19, 2023 (KR) 10-2023-0008194

(51) Int. Cl. G01N 27/12; G01N 33/00

(52) U.S. Cl. CPC G01N 27/127; G01N 27/123; G01N 27/126; G01N 33/005

(57) ABSTRACT

A resistive hydrogen sensor comprising a sensing layer having semiconducting single-walled carbon nanotubes (SWCNTs), and a manufacturing method therefor are disclosed. The hydrogen sensor comprises: a substrate; a sensing layer, which is formed on the substrate and comprises semiconducting SWCNTs; and electrodes formed on the surface of the sensing layer in the direction opposite to the direction facing the substrate, or formed between the sensing layer and the substrate, and spaced from each other, and thus has a sensitivity of 5% or higher and a response time of two seconds or less with respect to a hydrogen gas having a hydrogen concentration of 4 vol % in comparison to when there is no hydrogen, and can be operated at a low temperature.

### TECHNICAL FIELD

The present disclosure relates to a resistive hydrogen sensor including a sensing layer containing semiconducting single-walled carbon nanotubes and to a method of manufacturing the same.

### BACKGROUND ART

Recently, interest in eco-friendly and infinite hydrogen energy, the hydrogen economy, and efforts to establish the same are growing. Hydrogen is an inexhaustible, energetically reactive, and pollution-free energy source without smoke emission. However, hydrogen, a colorless, odorless, and tasteless flammable gas, is undetectable by human senses and may explode when 4% or more thereof leaks into the air. For this reason, there is a need to sense a hydrogen gas leak and measure quantified concentration to ensure safety.

Currently available methods of detecting hydrogen include gas chromatography, mass spectrometers, specific ionization gas pressure sensors, etc. Nevertheless, these methods have limitations. For example, the apparatuses are bulky, require high maintenance costs, and take a long time for sampling and measurement. Accordingly, while research has been conducted on detecting hydrogen gas through various chemical, electrical, and optical methods, resistive hydrogen sensors based on catalytic combustion methods using metal-oxide-semiconductors have achieved commercial success. However, catalytic combustion requires high temperatures, and achieving low power consumption and miniaturization are challenging. Resistive hydrogen sensors using metal-oxide-semiconductors have somewhat poor hydrogen selectivity, require oxygen for operation, and are highly sensitive to oxygen concentrations. Furthermore, while the response time ranges from about 4 to 20 seconds, a further improved response time (shorter than 2 seconds in the field of hydrogen vehicles) is required for use in the fields of hydrogen vehicles and the like.

However, there is a problem in that no technology for new semiconductor-based sensing materials and hydrogen sensors using the same to meet the requirements of these sensors exists.

### DISCLOSURE

### Technical Problem

The present disclosure has been made to solve the problems described above and aims to provide a high-performance hydrogen sensor and a method of manufacturing the same, the sensor having a fast response time of 2 seconds or less even when exposed to a small amount of hydrogen gas.

Additionally, the present disclosure aims to provide a high-performance hydrogen sensor and a method of manufacturing the same, the sensor not only having excellent sensing capabilities but also being commercializable with ease.

### Technical Solution

According to one aspect of the present disclosure, provided is a hydrogen sensor 10 for detecting hydrogen gas, which includes: a substrate 100; a sensing layer 200 formed on the substrate 100 and containing semiconducting single-wall carbon nanotubes (SWCNTs) 210; and electrodes 300 spaced from each other, the electrodes 300 formed on a first surface of the sensing layer 200, the first surface being a surface opposite to and not facing the substrate 100, or formed between the sensing layer 200 and the substrate 100.

Additionally, the sensing layer 200 may further contain a conjugated polymer 220, in which the surface of the semiconducting single-walled carbon nanotubes 210 may be partially or entirely wrapped with the conjugated polymer 220.

Additionally, the conjugated polymer 220 may include one or more selected from the group consisting of polyfluorene, 1,4-diketopyrrolo [3,4-c]pyrrole (DPP), naphthalene diimide, naphthalene-bis(dicarboximide) (NDI), isoindigo, isothiophene indigo, benzodipyrrolidone (BPT), poly(9,9-di-n-dodecylfluorene) (PFDD), poly(3-dodecylthiophene-2,5-diyl) (P3DDT), and poly(3-hexylthiophene-2,5-diyl) (P3HT).

Additionally, the surface of the hydrogen sensor 10 may be treated with one or more selected from the group consisting of ozone, ultraviolet, a surfactant, a self-assembled monolayer (SAM), a polymer coating, and a plasma and is, preferably, treated with ozone.

Additionally, the hydrogen sensor 10 may include a catalyst layer 400 positioned on the sensing layer 200 and the electrodes 300, the catalyst layer 400 containing a catalyst.

Additionally, the catalyst may include one or more selected from the group consisting of palladium (Pd), platinum (Pt), nickel (Ni), titanium (Ti), cobalt (Co), copper (Cu), and iron (Fe).

Additionally, the hydrogen sensor 10 may further include a first insulating layer 500 positioned on the sensing layer 200 and the electrodes 300, the first insulating layer 500 containing a first insulator.

Additionally, the first insulator may include one or more selected from the group consisting of parylene, polymethyl methacrylate (PMMA), polyvinyl phenol (PVP), polyvinyl alcohol (PVA), polystyrene (PS), polyimide (PI), polycarbonate (PC), and PEN, PET, Cytop, PTFE, SiO₂, HfOₓ, Al₂O₃, TiO, and SiNₓ.

Additionally, the hydrogen sensor 10 may further include a second insulating layer 600 and a microheater 700, in which the second insulating layer 600 may contain a second insulator and be positioned on the substrate 100 and under the sensing layer 200 and the electrodes 300, and the microheater 700 may be positioned between the substrate 100 and the second insulating layer 600.

Additionally, the second insulator may include one or more selected from the group consisting of parylene, polymethyl methacrylate (PMMA), polyvinyl phenol (PVP), polyvinyl alcohol (PVA), polystyrene (PS), polyimide (PI), polycarbonate (PC), and PEN, PET, Cytop, PTFE, SiO₂, HfOₓ, Al₂O₃, TiO, and SiNₓ.

Additionally, the substrate 100 may include one or more selected from the group consisting of a silicon (Si) substrate, a silicon/silicon dioxide (Si/SiO₂) substrate, a silicon/silicon nitride (Si/SiNₓ) substrate, a glass substrate, polyethylene terephthalate (PET), polyimide (PI), polyethylene naphthalate (PEN), polyethersulfone (PES), polyacrylate, and polyetherimide.

Additionally, the electrodes 300 may contain one or more selected from the group consisting of a metal, an oxide, a conductive polymer, and a carbon compound.

Additionally, the hydrogen sensor may be a resistive hydrogen sensor in which a resistance value of the sensing layer changes in the presence of hydrogen gas.

According to another aspect of the present disclosure, provided is a method of manufacturing a hydrogen sensor 10 for detecting hydrogen, which includes: (a) providing a substrate 100; (b) forming a sensing layer 200 by coating the substrate 100 with a solution containing semiconducting single-walled carbon nanotubes 210; and (c) manufacturing a hydrogen sensor by forming electrodes 300 to be spaced from each other, the electrodes 300 formed on a first surface of the sensing layer 200, the first surface being a surface opposite to and not facing the substrate 100, or formed between the sensing layer 200 and the substrate 100.

Additionally, before the (b) forming, the method of manufacturing the hydrogen sensor may further include: (b′-1) preparing a first mixture by mixing a conjugated polymer and unrefined single-walled carbon nanotubes containing metallic and semiconducting single-walled carbon nanotubes; (b′-2) preparing a second mixture containing conjugated polymer-wrapped semiconducting single-walled carbon nanotubes by sonicating the first mixture such that the surface of the semiconducting single-walled carbon nanotubes selected from among the metallic and semiconducting single-walled carbon nanotubes is wrapped with the conjugated polymer; and (b′-3) separating the conjugated polymer-wrapped semiconducting single-walled carbon nanotubes from the second mixture.

Additionally, after the (b′-3) separating, the method of manufacturing the hydrogen sensor may further include (b′-4) obtaining the semiconducting single-walled carbon nanotubes by removing the conjugated polymer from the conjugated polymer-wrapped semiconducting single-walled carbon nanotubes.

Additionally, the unrefined single-walled carbon nanotubes may be prepared by one selected from the group consisting of a plasma growth method, a high-pressure carbon monoxide (HiPco) method, an electric arc-discharge method, a laser vaporization method, a thermal chemical vapor deposition method, and a vapor-phase growth method.

Additionally, after the (c) manufacturing, the method of manufacturing the hydrogen sensor may further include (d-1) treating the surface of the hydrogen sensor with one or more selected from the group consisting of ultraviolet, ozone, a surfactant, a self-assembled monolayer (SAM), a polymer coating, and a plasma.

Additionally, after the (c) manufacturing, the method of manufacturing the hydrogen sensor may further include (d-2) forming a catalyst layer positioned on the sensing layer and the electrodes, the catalyst layer containing a catalyst.

Additionally, after the (c) manufacturing, the method of manufacturing the hydrogen sensor may further include (d-3) forming a first insulating layer positioned on the sensing layer and the electrodes, the first insulating layer containing a first insulator.

Additionally, after the (a) providing, the method of manufacturing the hydrogen sensor may further include (a′) positioning a microheater on the substrate and then forming a second insulating layer containing a second insulator on the microheater.

Additionally, the catalyst may include one or more selected from the group consisting of palladium (Pd), platinum (Pt), nickel (Ni), titanium (Ti), cobalt (Co), copper (Cu), and iron (Fe).

According to a further aspect of the present disclosure, provided is a method of detecting hydrogen, which includes: (1) bringing a gas in which hydrogen is required to be detected into contact with a sensing layer of the hydrogen sensor; and (2) detecting hydrogen gas by confirming a change in resistance or current value of the hydrogen sensor.

Additionally, the (1) bringing may be performed in the air.

### Advantageous Effects

A hydrogen sensor of the present disclosure has a form in which a sensing layer contains semiconducting single-walled carbon nanotubes (SWCNTs), thereby having a sensitivity of 5% or higher and a fast response time of 2 seconds or less and being operable at low temperatures.

Additionally, a method of manufacturing a hydrogen sensor of the present disclosure enables a sensing layer containing semiconducting single-walled carbon nanotubes (SWCNTs) to be formed on flexible substrates, such as plastic, through a low-temperature solution process and can improve price competitiveness by reducing manufacturing costs through a printing process.

### DESCRIPTION OF DRAWINGS

These are for the purpose of describing exemplary embodiments of the present disclosure, and therefore the technical idea of the present disclosure should not be construed as being limited to the accompanying drawings:

FIG. 1A schematically illustrates a hydrogen sensor (top-contact structure) according to one example of the present disclosure;

FIG. 1B schematically illustrates a hydrogen sensor (bottom-contact structure) according to one example of the present disclosure;

FIG. 2A schematically illustrates a hydrogen sensor (top-contact structure) including a catalyst layer 400 or a first insulating layer 500 according to one example of the present disclosure;

FIG. 2B schematically illustrates a hydrogen sensor (bottom-contact structure) including a catalyst layer 400 or a first insulating layer 500 according to one example of the present disclosure;

FIG. 3A schematically illustrates a hydrogen sensor (top-contact structure) including a second insulating layer 600 and microheaters 700 according to one example of the present disclosure;

FIG. 3B schematically illustrates a hydrogen sensor (bottom-contact structure) including a second insulating layer 600 and microheaters 700 according to one example of the present disclosure;

FIG. 4 schematically illustrates semiconducting single-walled carbon nanotubes wrapped with a conjugated polymer used in a sensing layer in a hydrogen sensor according to one example of the present disclosure;

FIG. 5 shows actual images of hydrogen sensors manufactured according to Examples 2 and 3;

FIG. 6 shows changes in the electrical properties of a hydrogen sensor manufactured according to Example 1 with the presence or absence of hydrogen gas;

FIG. 7 shows results of confirming changes in the electrical properties of a hydrogen sensor manufactured according to Example 2 with the presence or absence of hydrogen gas;

FIG. 8 schematically shows results of measuring the response time of a hydrogen sensor manufactured according to Example 2 with respect to hydrogen gas;

FIG. 9 shows results of confirming changes in the electrical properties of a hydrogen sensor manufactured according to Example 3 with the presence or absence of hydrogen gas;

FIG. 10 schematically shows results of measuring the response time of a hydrogen sensor manufactured according to Example 1 with respect to hydrogen gas;

FIG. 11 shows changes in the electrical properties of a hydrogen sensor (without membrane) manufactured according to Example 1 and a hydrogen sensor (with membrane) manufactured according to Example 4, with the presence or absence of hydrogen gas;

FIG. 12 shows results of measuring the sensitivity of a hydrogen sensor (without membrane) manufactured according to Example 1 and a hydrogen sensor (with membrane) manufactured according to Example 4, with respect to interfering gases (CO and CH₄); and

FIG. 13 shows changes in the electrical properties of a hydrogen sensor manufactured according to Example 5 with or without heating a substrate using a microheater.

### BEST MODE

Hereinafter, embodiments of the present disclosure will be described in detail with reference to the accompanying drawings so that those skilled in the art can easily carry out the present disclosure.

However, the following description does not limit the present disclosure to specific embodiments. In the following description of the present disclosure, the detailed description of related arts will be omitted if it is determined that the gist of the present disclosure may be blurred.

Terms used herein are used only to describe specific embodiments and are not intended to limit the present disclosure. The singular expression includes the plural expression unless the context clearly indicates otherwise. It will be further understood that the terms “comprises”, “includes”, or “has” when used herein specify the presence of stated features, integers, regions, steps, operations, elements, and/or components, but do not preclude the presence or addition of one or more other features, regions, integers, steps, operations, elements, components, and/or combinations thereof.

In addition, terms used in the specification, “first”, “second”, etc., may be used to describe various components, but the components are not to be construed as being limited to the terms. These terms are used only for the purpose of distinguishing a component from another component. For example, without departing from the scope of the present disclosure, a first component may be referred to as a second component, and a second component may be also referred to as a first component.

In addition, when a component is referred to as being “formed” or “stacked” on another component, it may be formed directly or attached to the front or one surface on the surface of the other component, but it will be understood that intervening elements may be present therebetween.

Hereinafter, a resistive hydrogen sensor including a sensing layer containing semiconducting single-walled carbon nanotubes and a method of manufacturing the same will be described in detail. However, these are disclosed only for illustrative purposes, by which the present disclosure is not limited, and the scope of the present disclosure is only defined by the appended claims.

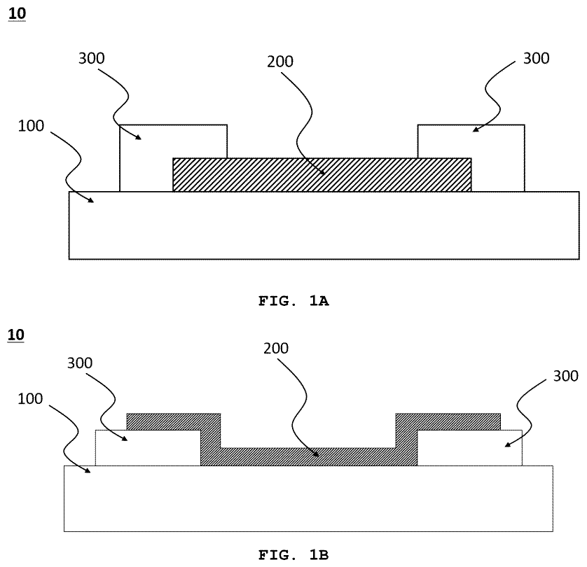

FIG. 1A schematically illustrates a hydrogen sensor (top-contact structure) according to one example of the present disclosure, and FIG. 1B schematically illustrates a hydrogen sensor (bottom-contact structure) according to one example of the present disclosure.

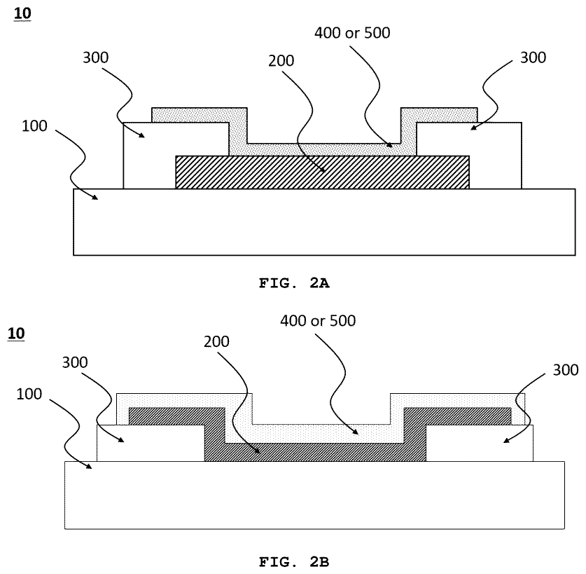

FIG. 2A schematically illustrates a hydrogen sensor (top-contact structure) including a catalyst layer 400 or a first insulating layer 500, according to one example of the present disclosure, and FIG. 2B schematically illustrates a hydrogen sensor (bottom-contact structure) including a catalyst layer 400 or a first insulating layer 500, according to one example of the present disclosure.

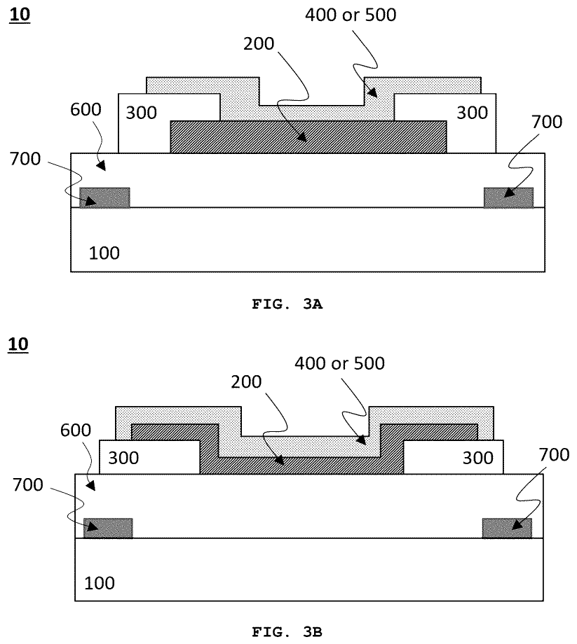

FIG. 3A schematically illustrates a hydrogen sensor (top-contact structure) including a second insulating layer 600 and microheaters 700, according to one example of the present disclosure, and FIG. 3B schematically illustrates a hydrogen sensor (bottom-contact structure) including a second insulating layer 600 and microheaters 700, according to one example of the present disclosure.

Referring to FIGS. 1A to 3B, the present disclosure provides a hydrogen sensor 10 for detecting hydrogen gas, which includes: a substrate 100; a sensing layer 200 formed on the substrate 100 and containing semiconducting single-walled carbon nanotubes (SWCNTs) 210; and electrodes 300 spaced from each other, the electrodes 300 formed on a first surface of the sensing layer 200, the first surface being a surface opposite to and not facing the substrate 100, or formed between the sensing layer 200 and the substrate 100.

When the electrodes 300 are formed on the first surface of the sensing layer 200, the surface opposite to and not facing the substrate 100, it is called a top-contact structure (see FIGS. 1A, 2A, and 3A), and when the electrodes 300 are formed between the sensing layer 200 and the substrate 100, it is called a bottom-contact structure (see FIGS. 1B, 2B, and 3B). Additionally, to be in more detail, when the electrodes 300 are formed on a part or all of the first surface of the sensing layer 200, the surface opposite to and not facing the substrate 100, it is called the top contact structure (see FIGS. 1A, 2A, and 3A), and when the electrodes 300 are formed between the substrate 100 and a part or all of the sensing layer 200, it is called the bottom-contact structure (see FIGS. 1B, 2B, and 3B).

Additionally, the sensing layer 200 may further contain a conjugated polymer 220, in which the surface of the semiconducting single-walled carbon nanotube 210 may be partially or entirely wrapped with the conjugated polymer 220.

Additionally, the conjugated polymer 220 may include one or more selected from the group consisting of polyfluorene, 1,4-diketopyrrolo [3,4-c]pyrrole (DPP), naphthalene diimide, naphthalene-bis(dicarboximide) (NDI), isoindigo, isothiophene indigo, benzodipyrrolidone (BPT), poly(9,9-di-n-dodecylfluorene) (PFDD), poly(3-dodecylthiophene-2,5-diyl) (P3DDT), and poly(3-hexylthiophene-2,5-diyl) (P3HT).

Additionally, the surface of the hydrogen sensor 10 may be treated with one or more selected from the group consisting of ozone, ultraviolet, a surfactant, a self-assembled monolayer (SAM), a polymer coating, and a plasma and is, preferably, treated with ozone.

Such surface treatment may be performed on the entire hydrogen sensor 10 and is, preferably, performed on the sensing layer 200 of the hydrogen sensor 10.

Additionally, the hydrogen sensor 10 may include a catalyst layer 400 positioned on the sensing layer 200 and the electrodes 300, the catalyst layer 400 containing a catalyst.

Additionally, the catalyst may include one or more selected from the group consisting of palladium (Pd), platinum (Pt), nickel (Ni), titanium (Ti), cobalt (Co), copper (Cu), and iron (Fe) and, preferably, includes palladium.

Additionally, the hydrogen sensor 10 may further include a first insulating layer 500 positioned on the sensing layer 200 and the electrodes 300, the first insulating layer 500 containing a first insulator.

Additionally, the first insulator may include one or more selected from the group consisting of parylene, polymethyl methacrylate (PMMA), polyvinyl phenol (PVP), polyvinyl alcohol (PVA), polystyrene (PS), polyimide (PI), polycarbonate (PC), and PEN, PET, Cytop, PTFE, SiO₂, HfOₓ, Al₂O₃, TiO, and SiNₓ and, preferably, includes Cytop.

Additionally, the first insulating layer 500 may be formed by stacking two or more types of insulation materials.

Additionally, the hydrogen sensor 10 may further include a second insulating layer 600 and a microheater 700, in which the second insulating layer 600 may contain a second insulator and be positioned on the substrate 100 and under the sensing layer 200 and the electrodes 300, and the microheater 700 may be positioned between the substrate 100 and the second insulating layer 600.

Additionally, the second insulator may include one or more selected from the group consisting of parylene, polymethyl methacrylate (PMMA), polyvinyl phenol (PVP), polyvinyl alcohol (PVA), polystyrene (PS), polyimide (PI), polycarbonate (PC), and PEN, PET, Cytop, PTFE, SiO₂, HfOₓ, Al₂O₃, TiO, and SiNₓ and, preferably, includes SiO₂.

Additionally, the second insulating layer 500 may be formed by stacking two or more types of insulation materials.

The microheater mainly aims to provide an environment where a sensor is stably operable regardless of external temperature changes by keeping the temperature in the hydrogen sensor, including the sensing layer containing the carbon nanotubes, constant. Additionally, the microheater may improve sensitivity and actively enable compensation for the temperature.

The temperature of the substrate is preferably set in consideration of the operating environment of the hydrogen sensor. Typically, the temperature of the substrate may be in a range of 30° C. to 100° C. and, more preferably, in the range of 50° C. to 80° C. When the temperature of the substrate is lower than 30° C., the device is likely to be affected by changes in higher external temperatures (for example, in the summertime, tropical regions, and the like), which is undesirable. When the temperature of the substrate exceeds 100° C., there may be problems with stability and power consumption, which is undesirable.

Additionally, the substrate 100 may include one or more selected from the group consisting of a silicon (Si) substrate, a silicon/silicon dioxide (Si/SiO₂) substrate, a silicon/silicon nitride (Si/SiNₓ) substrate, a glass substrate, polyethylene terephthalate (PET), polyimide (PI), polyethylene naphthalate (PEN), polyethersulfone (PES), polyacrylate, and polyetherimide.

Additionally, the electrodes 300 may contain one or more selected from the group consisting of a metal, an oxide, a conductive polymer, and a carbon compound.

Specifically, the electrodes 300 may contain one or more selected from the group consisting of gold (Au), silver (Ag), platinum (Pt), titanium (Ti), aluminum (Al), tungsten (W), magnesium (Mg), calcium (Ca), ytterbium (Yb), chromium (Cr), nickel (Ni), gold oxide, platinum oxide, silver oxide, palladium oxide, iron oxide, graphene, carbon nanotubes (CNTs), silver nanowire (Ag nanowire, Ag NW), indium tin oxide, and poly(3,4-ethylenedioxythiophene) polystyrene sulfonate (PEDOT: PSS). Preferably, there is no limitation in the materials of the electrodes to be developed in the future.

Additionally, the hydrogen sensor may detect hydrogen gas in the air.

Additionally, the hydrogen sensor may be a resistive hydrogen sensor in which the resistance value of the sensing layer changes in the presence of hydrogen gas.

The present disclosure provides a method of manufacturing a hydrogen sensor 10 for detecting hydrogen, which includes: (a) providing a substrate 100; (b) forming a sensing layer 200 by coating the substrate 100 with a solution containing semiconducting single-walled carbon nanotubes 210; and (c) manufacturing a hydrogen sensor by forming electrodes 300 to be spaced from each other, the electrodes 300 formed on a first surface of the sensing layer 200, the first surface being a surface opposite to and not facing the substrate 100, or formed between the sensing layer 200 and the substrate 100.

Additionally, before the (b) forming, the method of manufacturing the hydrogen sensor may further include: (b′-1) preparing a first mixture by mixing a conjugated polymer and unrefined single-walled carbon nanotubes containing metallic and semiconducting single-walled carbon nanotubes; (b′-2) preparing a second mixture containing conjugated polymer-wrapped semiconducting single-walled carbon nanotubes by sonicating the first mixture such that the surface of the semiconducting single-walled carbon nanotubes selected from among the metallic and semiconducting single-walled carbon nanotubes is wrapped with the conjugated polymer; and (b′-3) separating the conjugated polymer-wrapped semiconducting single-walled carbon nanotubes from the second mixture.

Additionally, after the (b′-3) separating, the method of manufacturing the hydrogen sensor may further include (b′-4) obtaining the semiconducting single-walled carbon nanotubes by removing the conjugated polymer from the conjugated polymer-wrapped semiconducting single-walled carbon nanotubes.

Additionally, the unrefined single-walled carbon nanotubes may be prepared by one selected from the group consisting of a plasma growth method, a high-pressure carbon monoxide (HiPco) method, an electric arc-discharge method, a laser vaporization method, a thermal chemical vapor deposition method, and a vapor-phase growth method and are, preferably, prepared by the plasma growth method.

Additionally, after the (a) providing, the method of manufacturing the hydrogen sensor may further include (a″) modifying a part or all of the surface of the substrate with one selected from the group consisting of a surfactant (HMDS and the like), a self-assembled monolayer (SAM), a polymer coating, plasma treatment, and ozone treatment.

Additionally, after the (c) manufacturing, the method of manufacturing the hydrogen sensor may further include (d-1) treating the surface of the hydrogen sensor with one or more selected from the group consisting of ultraviolet, ozone, a surfactant, a self-assembled monolayer (SAM), a polymer coating, and a plasma.

Such surface treatment may be performed on the entire hydrogen sensor and is, preferably, performed on the sensing layer of the hydrogen sensor.

Additionally, after the (c) manufacturing, the method of manufacturing the hydrogen sensor may further include (d-2) forming a catalyst layer positioned on the sensing layer and the electrodes, the catalyst layer containing a catalyst.

Additionally, after the (c) manufacturing, the method of manufacturing the hydrogen sensor may further include (d-3) forming a first insulating layer positioned on the sensing layer and the electrodes, the first insulating layer containing a first insulator.

Additionally, after the (a) providing, the method of manufacturing the hydrogen sensor may further include (a′) positioning a microheater on the substrate and then forming a second insulating layer containing a second insulator on the microheater.

Additionally, the catalyst may include one or more selected from the group consisting of palladium (Pd), platinum (Pt), nickel (Ni), titanium (Ti), cobalt (Co), copper (Cu), and iron (Fe) and, preferably, includes palladium.

The present disclosure provides a method of detecting hydrogen, which includes: (1) bringing a gas in which hydrogen is required to be detected into contact with a sensing layer of the hydrogen sensor; and (2) detecting hydrogen gas by confirming a change in resistance or current value of the hydrogen sensor.

Additionally, the (1) bringing may be performed in the air.

Additionally, when bringing the sensing layer into contact with hydrogen gas, a resistance value may change.

Additionally, before the (1) bringing, the method of detecting hydrogen may further include (1′) adjusting a temperature of the substrate in the hydrogen sensor.

The temperature of the substrate is preferably set in consideration of the operating environment of the hydrogen sensor. Typically, the temperature of the substrate may be in a range of 30° C. to 100° C. and, more preferably, in the range of 50° C. to 80° C. When the temperature of the substrate is lower than 30° C., the device is likely to be affected by changes in higher external temperatures (for example, in the summertime, tropical regions, and the like), which is undesirable. When the temperature of the substrate exceeds 100° C., there may be problems with stability and power consumption, which is undesirable.

### MODE FOR INVENTION

### Example

Hereinafter, preferred examples of the present disclosure will be described. However, these examples are disclosed for illustrative purposes and the scope of the present disclosure is not limited thereby.

### Manufacturing of Hydrogen Sensor

### Example 1: Hydrogen Sensor (not Involving Ozone Treatment and Pd Deposition)

FIG. 1A schematically illustrates a hydrogen sensor (top-contact structure) according to one example of the present disclosure, and FIG. 1B schematically illustrates a hydrogen sensor (bottom-contact structure) according to one example of the present disclosure. A hydrogen sensor in Example 1 was manufactured with reference to FIGS. 1A and 1B, such that the hydrogen sensor in Example 1 had a top-contact structure (FIG. 1A).

After mixing 5 mg of a mixture of metallic and semiconducting single-walled carbon nanotubes (plasma tubes SWCNTs, purchased from NanoIntegris), synthesized through a plasma growth method, and 10 mg of a conjugated polymer in 20 mL of toluene, the resulting mixture was sonicated and centrifuged to separate conjugated polymer-wrapped semiconducting single-walled carbon nanotubes. The polymer residues in the solution were removed using a filter, and CNT ink having a concentration of 0.05 mg/mL was ultimately prepared.

In this case, the single-walled carbon nanotubes, synthesized through a plasma growth method, had a diameter of 0.9 to 1.5 nm, a length of 0.3 to 4.0 μm, and an energy band gap of 0.5 to 0.9 eV.

Surface treatment was performed with bis(trimethylsilyl)amine (HMDS) on a part of the surface of a silicon substrate (Si/SiO₂) whose surface includes an oxide layer.

The substrate was coated with a solution containing the conjugated polymer-wrapped semiconducting single-walled carbon nanotubes to form a sensing layer containing the conjugated polymer-wrapped semiconducting single-walled carbon nanotubes on the part of the surface having been subjected to the surface treatment.

Thereafter, the hydrogen sensor was manufactured by forming a nickel (Ni)/gold (Au) electrode pattern through vacuum thermal deposition using a shadow mask.

### Example 2: Hydrogen Sensor Involving Ozone Treatment

FIG. 1A schematically illustrates a hydrogen sensor (top-contact structure) according to one example of the present disclosure, and FIG. 1B schematically illustrates a hydrogen sensor (bottom-contact structure) according to one example of the present disclosure. A hydrogen sensor in Example 2 was manufactured with reference to FIGS. 1A and 1B, such that the hydrogen sensor in Example 2 had a top-contact structure (FIG. 1A).

UV-O₃ surface treatment was performed on the hydrogen sensor, manufactured according to Example 1, for a predetermined period of time to complete the manufacturing of the hydrogen sensor having improved reactivity and response time.

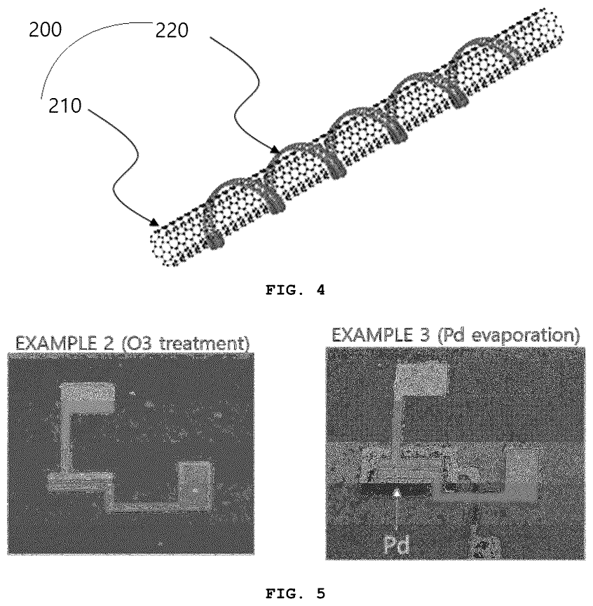

FIG. 5 confirms an image of the hydrogen sensor (involving O₃ treatment) manufactured according to Example 2.

### Example 3: Hydrogen Sensor Including Pd Catalyst Layer

FIG. 2A schematically illustrates a hydrogen sensor (top-contact structure) including a catalyst layer 400 or a first insulating layer 500, according to one example of the present disclosure, and FIG. 2B schematically illustrates a hydrogen sensor (bottom-contact structure) including a catalyst layer 400 or a first insulating layer 500, according to one example of the present disclosure. A hydrogen sensor in Example 3 was manufactured with reference to FIGS. 2A and 2B, such that the hydrogen sensor in Example 3 had a top-contact structure (FIG. 2A).

A catalyst layer was formed by depositing palladium (Pd) using a shadow mask on a sensing layer and an electrode pattern of the hydrogen sensor, manufactured according to Example 1, to manufacture the hydrogen sensor.

FIG. 5 confirms an image of the hydrogen sensor (involving Pd evaporation) manufactured according to Example 3.

### Example 4: Hydrogen Sensor Including First Insulating Layer

FIG. 2A schematically illustrates a hydrogen sensor (top-contact structure) including a catalyst layer 400 or a first insulating layer 500, according to one example of the present disclosure, and FIG. 2B schematically illustrates a hydrogen sensor (bottom-contact structure) including a catalyst layer 400 or a first insulating layer 500, according to one example of the present disclosure. A hydrogen sensor in Example 4 was manufactured with reference to FIGS. 2A and 2B, such that the hydrogen sensor in Example 4 had a top-contact structure (FIG. 2A).

A sensing layer of the hydrogen sensor manufactured according to Example 1 and an electrode pattern of the hydrogen sensor were coated with a polymer insulator (Cytop) to manufacture the hydrogen sensor further including an insulating layer.

### Example 5: Hydrogen Sensor Including Second Insulating Layer and Microheater

FIG. 3A schematically illustrates a hydrogen sensor (top-contact structure) including a second insulating layer 600 and microheaters 700, according to one example of the present disclosure, and FIG. 3B schematically illustrates a hydrogen sensor (bottom-contact structure) including a second insulating layer 600 and microheaters 700, according to one example of the present disclosure. A hydrogen sensor in Example 5 was manufactured with reference to FIGS. 3A and 3B, such that the hydrogen sensor in Example 5 had a top-contact structure (FIG. 3A).

When manufacturing the t hydrogen sensor according to Example 1, microheaters (Pt) were positioned on a silicon substrate (Si/SiO₂) including an oxide layer formed on the surface, and a step of forming a second insulating layer containing SiO₂ on the microheaters was further added to manufacture the hydrogen sensor including the second insulating layer and the microheaters.

### Experimental Example

### Experimental Example 1: Confirmation of Hydrogen Detection by Hydrogen Sensor

An experiment was conducted to confirm the hydrogen detection performance of the hydrogen sensors manufactured according to Examples 1 to 3. To be in more detail, the hydrogen detection performance was confirmed on the basis of changes in the electrical properties of the hydrogen sensors to which a voltage level of 3 V was applied when repeatedly injecting hydrogen gas mixed at 4 vol % with argon, an inert gas, thereinto.

In this case, the sensitivity of each hydrogen sensor was calculated according to Equation 1 below. Additionally, the response time of each hydrogen sensor, measured based on ISO 26142, refers to the time required for the hydrogen sensor to reach a stated percentage of the stabilized (saturated) signal for a specific concentration of hydrogen gas, which is typically the time (t90) required to reach 90% of the stable signal value.

$$Sensitivity ( \% ) = \frac{\vert {R}_{2} - {R}_{1} \vert}{{R}_{1}} \times 100 \qquad [ Equation 1 ]$$

In Equation 1 above,
R₁ is a resistance value or current value of the hydrogen sensor in the absence of hydrogen, and
R₂ is a resistance value or current value of the hydrogen sensor in the presence of hydrogen.

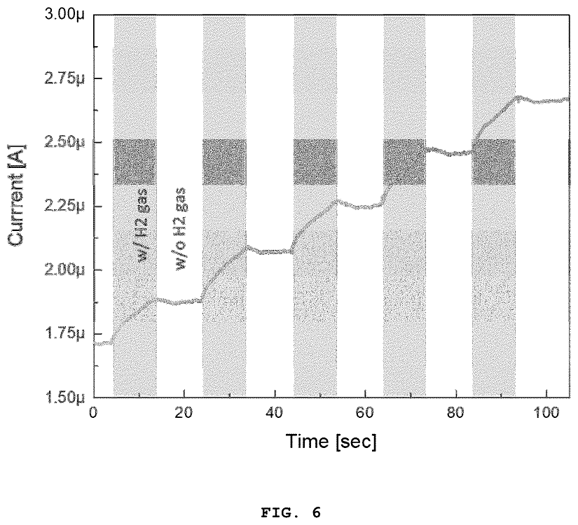

FIG. 6 confirms changes in the electrical properties of the hydrogen sensor, manufactured according to Example 1, with the presence or absence of hydrogen gas.

According to FIG. 6, the current value of the hydrogen sensor, manufactured according to Example 1, is confirmed to increase at a constant slope as the sensing layer of the hydrogen sensor reacts with air. Additionally, the current value is confirmed to increase (slope increases) when injecting hydrogen gas into the hydrogen sensor while decreasing (slope decreases) when stopping the injection of hydrogen gas.

Therefore, when using high-purity semiconducting single-walled carbon nanotubes, separated using the conjugated polymer, as the sensing layer, a hydrogen sensor capable of detecting hydrogen may be manufactured.

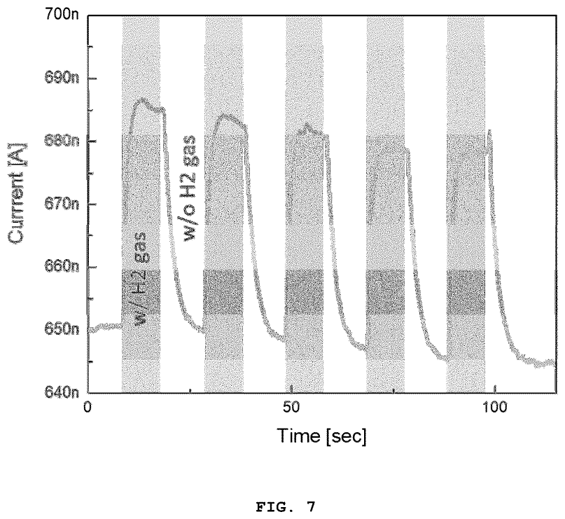

FIG. 7 confirms changes in the electrical properties of the hydrogen sensor, manufactured according to Example 2, with the presence or absence of hydrogen gas.

According to FIG. 7, the hydrogen sensor, manufactured according to Example 2, is confirmed to have a stable current value even in the air. Additionally, the hydrogen sensor also has a base current of 0.651 ρA and a sensitivity of 5.5% with respect to 4% hydrogen gas, confirming that the sensitivity thereof is superior that of the to hydrogen sensor manufactured according to Example 1, not involving ozone treatment.

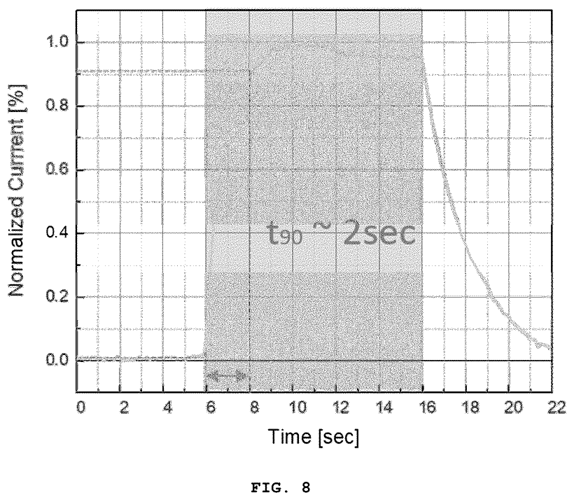

FIG. 8 confirms the response time of the hydrogen sensor, manufactured according to Example 2, with the presence or absence of hydrogen gas.

According to FIG. 8, the hydrogen sensor, manufactured according to Example 2, has a response time (t90) of about 2 seconds, confirming that the response time thereof is superior to that of the hydrogen sensor manufactured according to Example 1, not involving ozone treatment.

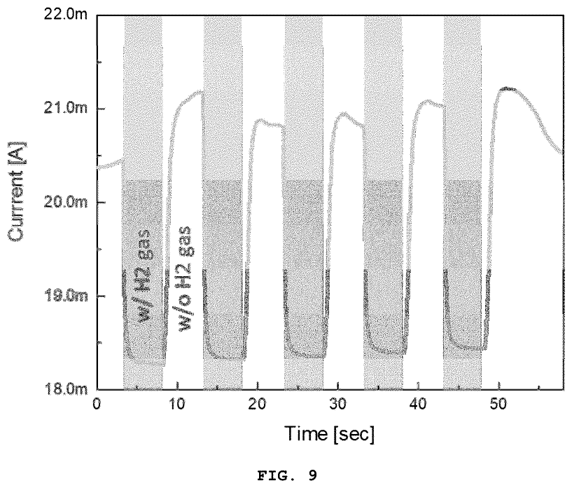

FIG. 9 confirms changes in the electrical properties of the hydrogen sensor, manufactured according to Example 3, with the presence or absence of hydrogen gas.

According to FIG. 9, the hydrogen sensor, manufactured according to Example 3, is confirmed to have a stable current value even in the air. Additionally, the hydrogen sensor also has a base current of 20.5 mA and a sensitivity of 10.6% with respect to 4% hydrogen gas, confirming that the sensitivity thereof is superior to that the hydrogen sensor manufactured according to Example 1, not involving palladium deposition.

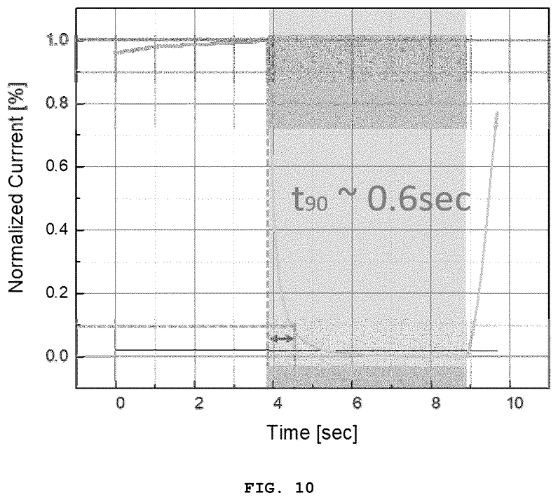

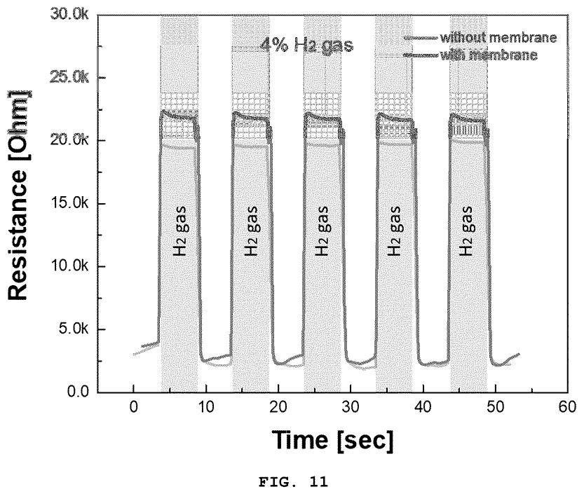

FIG. 10 confirms the response time of the hydrogen sensor, manufactured according to Example 3, with the presence or absence of hydrogen gas.

According to FIG. 10, the hydrogen sensor, manufactured according to Example 3, has a fast response time (t90) of about 0.6 seconds, confirming that the response time thereof is superior to that of the hydrogen sensor manufactured according to Example 1, not involving palladium deposition.

### Experimental Example 2: Confirmation of Reactivity of Hydrogen Gas and Interfering Gas with Presence or Absence of First Insulating Layer

FIG. 11 shows changes in the electrical properties of the hydrogen sensor (without membrane), manufactured according to Example 1, and the hydrogen sensor (with membrane), manufactured according to Example 4, with the presence or absence of hydrogen gas.

According to FIG. 11, the hydrogen sensor in Example 4, further including the first insulating layer, exhibits satisfactory properties with respect to hydrogen gas.

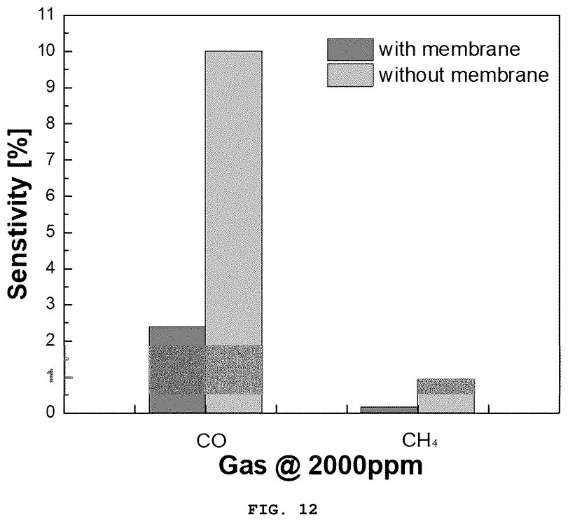

FIG. 12 shows the results of measuring the sensitivity of the hydrogen sensor (without membrane), manufactured according to Example 1, and the hydrogen sensor (with membrane), manufactured according to Example 4, to interfering gases (CO and CH₄).

According to FIG. 12, the hydrogen sensor in Example 4, further including the first insulating layer, exhibits low sensitivity to the interfering gases (CO and CH₄).

Therefore, when further including the first insulating layer, the hydrogen sensor of the present disclosure is confirmed to be able to exhibit satisfactory properties with respect to hydrogen while having low sensitivity to the interfering gases.

### Experimental Example 3: Confirmation of Hydrogen Gas Reactivity with or without Heating Substrate Using Microheater

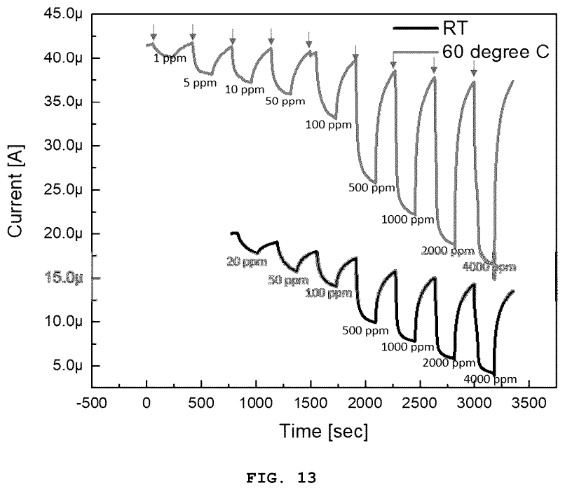

FIG. 13 shows changes in the electrical properties of the hydrogen sensor, manufactured according to Example 5, with or without heating the substrate using the microheaters.

According to FIG. 13, in the case where a temperature of 60° C. is applied to the substrate using the microheaters, the hydrogen sensor is confirmed to have better sensitivity and reactivity compared to the case where temperature is not additionally applied (RT).

The scope of the present disclosure is defined only by the appended claims rather than the detailed description. All changes or modifications derived from the meaning and scope of the claims and the concept of equivalents should be construed to fall within the scope of the present disclosure.

### EXPLANATION OF REFERENCE NUMERALS

10: Hydrogen sensor
100: Substrate
200: Sensing layer
210: Semiconducting single-walled carbon nanotube
220: Conjugated polymer
300: Electrode
400: Catalyst layer
500: First insulating layer
600: Second insulating layer
700: Microheater

### INDUSTRIAL APPLICABILITY

A hydrogen sensor of the present disclosure has a form in which a sensing layer contains semiconducting single-walled carbon nanotubes (SWCNTs), thereby having a sensitivity of 5% or higher and a fast response time of 2 seconds or less and being operable at low temperatures.

Additionally, a method of manufacturing a hydrogen sensor of the present disclosure enables a sensing layer containing semiconducting single-walled carbon nanotubes (SWCNTs) to be formed on flexible substrates, such as plastic, through a low-temperature solution process and may improve price competitiveness by reducing manufacturing costs through a printing process.

1. A hydrogen sensor for detecting hydrogen gas, the hydrogen sensor comprising:
a substrate;
a sensing layer formed on the substrate and containing semiconducting single-walled carbon nanotubes (SWCNTs); and
electrodes spaced from each other, the electrodes formed on a first surface of the sensing layer, the first surface being a surface opposite to and not facing the substrate, or formed between the sensing layer and the substrate.

2. The hydrogen sensor of claim 1, wherein the sensing layer further comprises a conjugated polymer, and
the surface of the semiconducting single-walled carbon nanotubes is partially or entirely wrapped with the conjugated polymer.

3. The hydrogen sensor of claim 2, wherein the conjugated polymer comprises one or more selected from the group consisting of polyfluorene, 1,4-diketopyrrolo [3,4-c]pyrrole (DPP), naphthalene diimide, naphthalene-bis(dicarboximide) (NDI), isoindigo, isothiophene indigo, benzodipyrrolidone (BPT), poly(9,9-di-n-dodecylfluorene) (PFDD), poly(3-dodecylthiophene-2,5-diyl) (P3DDT), and poly(3-hexylthiophene-2,5-diyl) (P3HT).

4. The hydrogen sensor of claim 1, wherein the surface of the hydrogen sensor is treated with one or more selected from the group consisting of ozone, ultraviolet, a surfactant, a self-assembled monolayer (SAM), a polymer coating, and a plasma.

5. The hydrogen sensor of claim 1, further comprising a catalyst layer positioned on the sensing layer and the electrodes, the catalyst layer comprising a catalyst.

6. The hydrogen sensor of claim 1, further comprising a first insulating layer positioned on the sensing layer and the electrodes, the first insulating layer comprising a first insulator.

7. The hydrogen sensor of claim 1, further comprising a second insulating layer and a microheater,
wherein the second insulating layer comprises a second insulator and is positioned on the substrate and under the sensing layer and the electrodes, and
the microheater is positioned between the substrate and the second insulating layer.

8. The hydrogen sensor of claim 1, wherein the substrate comprises one or more selected from the group consisting of a silicon (Si) substrate, a silicon/silicon dioxide (Si/SiO₂) substrate, a silicon/silicon nitride (Si/SiNₓ) substrate, a glass substrate, polyethylene terephthalate (PET), polyimide (PI), polyethylene naphthalate (PEN), polyethersulfone (PES), polyacrylate, and polyetherimide.

9. The hydrogen sensor of claim 1, wherein the electrodes comprise one or more selected from the group consisting of a metal, an oxide, a conductive polymer, and a carbon compound.

10. The hydrogen sensor of claim 1, wherein the hydrogen sensor is a resistive hydrogen sensor in which a resistance value of the sensing layer changes in the presence of hydrogen gas.

11. A method of manufacturing a hydrogen sensor for detecting hydrogen, the method comprising:
(a) providing a substrate;
(b) forming a sensing layer by coating the substrate with a solution comprising semiconducting single-walled carbon nanotubes; and
(c) manufacturing a hydrogen sensor by forming electrodes to be spaced from each other, the electrodes formed on a first surface of the sensing layer, the first surface being a surface opposite to and not facing the substrate, or formed between the sensing layer and the substrate.

12. The method of claim 11, further comprising:
(b′-1) preparing a first mixture by mixing a conjugated polymer and unrefined single-walled carbon nanotubes comprising metallic and semiconducting single-walled carbon nanotubes;
(b′-2) preparing a second mixture comprising conjugated polymer-wrapped semiconducting single-walled carbon nanotubes by sonicating the first mixture such that the surface of the semiconducting single-walled carbon nanotubes selected from among the metallic and semiconducting single-walled carbon nanotubes is wrapped with the conjugated polymer; and
(b′-3) separating the conjugated polymer-wrapped semiconducting single-walled carbon nanotubes from the second mixture, before the (b) forming.

13. The method of claim 12, further comprising (b′-4) obtaining the semiconducting single-walled carbon nanotubes by removing the conjugated polymer from the conjugated polymer-wrapped semiconducting single-walled carbon nanotubes, after the (b′-3) separating.

14. The method of claim 12, wherein the unrefined single-walled carbon nanotubes are prepared by one selected from the group consisting of a plasma growth method, a high-pressure carbon monoxide (HiPco) method, an electric arc-discharge method, a laser vaporization method, a thermal chemical vapor deposition method, and a vapor-phase growth method.

15. The method of claim 11, further comprising (d-1) treating the surface of the hydrogen sensor with one or more selected from the group consisting of ultraviolet, ozone, a surfactant, a self-assembled monolayer (SAM), a polymer coating, and a plasma, after the (c) manufacturing.

16. The method of claim 11, further comprising (d-2) forming a catalyst layer positioned on the sensing layer and the electrodes, the catalyst layer comprising a catalyst, after the (c) manufacturing.

17. The method of claim 11, further comprising (d-3) forming a first insulating layer positioned on the sensing layer electrodes, the first insulating layer comprising a first insulator, after the (c) manufacturing.

18. The method of claim 11, further comprising (a′) positioning a microheater on the substrate and then forming a second insulating layer comprising a second insulator on the microheater, after the (a) providing.

19. A method of detecting hydrogen, the method comprising:
(1) bringing a gas from which hydrogen is required to be detected into contact with a sensing layer of the hydrogen sensor of claim 1; and
(2) detecting hydrogen gas by confirming a change in resistance or current value of the hydrogen sensor.

20. The method of claim 19, wherein the (1) bringing is performed in the air.
